(19) United States
(12) Patent Application Publication
(10) Pub. No.: US 2013/0103154 A1
(43) Pub. Date: Apr. 25, 2013

(54) EXPANDABLE SPINAL IMPLANT AND METHODS OF USE

(71) Applicants: Hai H. Trieu, Cordova, TN (US); Keith E. Miller, Germantown, TN (US)

(72) Inventors: Hai H. Trieu, Cordova, TN (US); Keith E. Miller, Germantown, TN (US)

(73) Assignee: WARSAW ORTHOPEDIC, INC, Warsaw, IN (US)

(21) Appl. No.: 13/280,090

(22) Filed: Oct. 24, 2011

(51) Int. Cl. A61F 2/44

(52) U.S. Cl. 623/17.16

(57) ABSTRACT

A spinal implant includes first and second vertebral endplate-engaging components connected to each other at one end in articulated engagement to provide a desired expanded configuration, and an expander component disposed between the first and second components at the other end thereof to maintain the expanded configuration. Methods of use are disclosed.

### TECHNICAL FIELD

The present disclosure generally relates to medical implants and methods for the treatment of musculoskeletal disorders, and more particularly to an expandable spinal implant and related methods that provide stabilization and height restoration for treating a vertebral column.

### BACKGROUND

Spinal disorders such as degenerative disc disease, disc herniation, osteoporosis, spondylolisthesis, stenosis, scoliosis and other curvature abnormalities, kyphosis, tumor, and fracture may result from factors including trauma, disease and degenerative conditions caused by injury and aging. Spinal disorders typically result in symptoms including pain, nerve damage, and partial or complete loss of mobility. For example, after a disc collapse, severe pain and discomfort can occur due to the pressure exerted on nerves and the spinal column.

Non-surgical treatments, such as medication, rehabilitation and exercise can be effective, however, may fail to relieve the symptoms associated with these disorders. Surgical treatment of these spinal disorders includes fusion, fixation, discectomy, laminectomy and implantable prosthetics. These treatments may employ interbody implants. This disclosure describes an improvement over these prior art technologies.

### SUMMARY

Accordingly, an expandable spinal implant and related methods are provided that provide stabilization and height restoration for treating a vertebral column. It is contemplated that the expandable spinal implant is adjustable between a first, collapsed configuration and a second, expanded configuration. It is further contemplated that the implant and methods may be employed for an arthrodesis treatment using minimally invasive and percutaneous techniques.

In one embodiment, an expandable spinal implant is provided. The expandable spinal implant comprises first component comprising outer and inner surfaces. A first terminal section is disposed at one end of the first component and a second terminal section at its other end. The first component defines a lateral slot within the inner surface. A second component comprises outer and inner surfaces. A first terminal section is disposed at one end of the second component and a second terminal section at its other end. The inner surface of the second component defines a lateral slot. The first terminal section of the first component being connected with the first terminal section of the second component for substantially vertical arcuate movement of the first component relative to the second component. The range of such movement providing an expandable distance between the second terminal sections of the first and second components. The first terminal sections of the first and second components define a posterior face of the implant and the second terminal sections of the first and second components define a anterior face of the implant. An expander component is disposable within the lateral slots of the first and second components in a configuration for maintaining a predetermined distance between the second terminal sections of the first and second components.

In one embodiment, a lordosing implant is provided. The lordosing implant comprises an upper component comprising an outer, vertebral end plate-engaging surface, an inner surface defining a lateral cavity, a posterior section at one end of the component and an anterior section at the other end thereof. A lower component comprises an outer, vertebral endplate-engaging surface, an inner surface facing the inner surface of the upper component, the inner surface of the lower component defining a lateral cavity, the inner surfaces further defining a cavity therebetween, a posterior section at one end of the component and an anterior section at the other end thereof. The posterior section of the upper component being connected by a flexible member to the posterior section of the lower component for substantially vertical arcuate movement of the upper component relative to the lower component. The range of such movement extending from a first, collapsed configuration of the upper and lower components to a second, lordosing configuration of the upper and lower components. An expander component is disposable within the lateral cavities. The implant is disposable within an intervertebral space such that the lateral cavities are aligned along a lateral pathway of a body such that the expander component maintains a predetermined distance between the anterior sections corresponding to a desired lordosing configuration of the upper and lower components.

In one embodiment, a method for treating a spine is provided. The method comprises the steps of: providing an expandable spinal implant portion comprising: a first component comprising outer and inner surfaces, a first terminal section at one end of the first component and a second terminal section at its other end; and a second component comprising outer and inner surfaces, a first terminal section at one end of the second component and a second terminal section at its other end, the first terminal section of the upper component being connected in articulated engagement with the first terminal section of the lower component for substantially vertical arcuate movement of the first component relative to the second component, the range of such movement providing an expandable distance between the second terminal sections of the first and second components; introducing the expandable spinal implant portion along a direct lateral pathway of a body within an intervertebral space; and introducing an expander component along the direct lateral pathway between the second terminal sections of the first and second components to expand the distance between the second terminal sections of the first and second components of the expandable spinal implant portion by a predetermined distance, the expander component being sized for maintaining the predetermined distance between the second terminal portions of the first and second components.

### BRIEF DESCRIPTION OF THE DRAWINGS

The present disclosure will become more readily apparent from the specific description accompanied by the following drawings, in which:

FIGS. 1A and 1B are, respectively, side elevation and top plan views of a portion of one embodiment of an expandable spinal implant in accordance with the principles of the present disclosure, the implant portion being shown in the collapsed configuration;

FIGS. 2A and 2B are, respectively, perspective and front elevation views of the implant portion shown in FIGS. 1A and 1B in the collapsed configuration;

FIGS. 3A and 3B are, respectively, side elevation and front elevation views of the implant portion shown in FIGS. 2A and 2B in an expanded configuration with its expander component in place;

FIG. 4 is a side elevation view of one embodiment of an implant portion in accordance with the principles of the present disclosure;

FIG. 5 is a side view of vertebrae and a component of a spinal implant insertion system in accordance with the principles of the present disclosure;

FIG. 6 is a side view of the vertebrae and components of the implant insertion system shown in FIG. 5;

FIG. 7 is a side view of the vertebrae and components of the implant insertion system shown in FIG. 5 including an implant portion in the collapsed configuration as shown in FIGS. 1A and 1B as it is being inserted within an intervertebral space;

FIG. 8 is a side view of the vertebrae and the implant portion in the collapsed configuration shown in FIG. 7 positioned within the intervertebral space; and

FIG. 9 is a side view of the vertebrae and the expanded spinal implant as shown in FIGS. 3A and 3B.

### DETAILED DESCRIPTION

The exemplary embodiments of the expandable spinal implant and related methods of use disclosed herein are discussed in terms of medical devices for the treatment of musculoskeletal disorders and more particularly, in terms of an intervertebral implant that provides stabilization and height restoration for treating a vertebral column. It is envisioned that the expandable spinal implant may be employed for fusion and fixation treatments to provide decompression, restoration of lordosis and resistance of subsidence into tissue, for example, vertebral endplates. It is further envisioned that the expandable spinal implant and methods of use disclosed herein can be employed to obtain fusion of vertebrae through a minimally invasive or percutaneous technique. In one embodiment, the disclosed expandable spinal implant and methods of use can provide improved spinal treatment with a device that is made to expand vertically and in so doing to create lordosis in vertebrae. It is contemplated that the expandable spinal implant and methods of use disclosed herein provide anti-migration and locking of the implant in a predetermined lordosing configuration after expansion. It is further contemplated that the expandable spinal implant and methods of use disclosed herein provide a cavity of relatively large volume for post-packing of at least one agent, for example, bone graft.

It is envisioned that the present disclosure may be employed to treat spinal disorders such as, for example, degenerative disc disease, disc herniation, osteoporosis, spondylolisthesis, stenosis, scoliosis and other curvature abnormalities, kyphosis, tumor and fractures. It is contemplated that the present disclosure may be employed with other osteal and bone related applications, including those associated with diagnostics and therapeutics. It is further contemplated that the disclosed expandable spinal implant may be alternatively employed in a surgical treatment with a patient in a prone or supine position, and/or employ various surgical approaches to the spine, including anterior, posterior, posterior mid-line, medial, lateral, postero-lateral, and/or antero-lateral approaches, and in other body regions. The expandable spinal implant of the present disclosure may also be alternatively employed with procedures for treating the lumbar, cervical, thoracic and pelvic regions of a spinal column. The expandable spinal implant and methods of the present disclosure may also be used on animals, bone models and other non-living substrates, such as, for example, in training, testing and demonstration.

The present disclosure may be understood more readily by reference to the following detailed description of the disclosure taken in connection with the accompanying drawing figures, which form a part of this disclosure. It is to be understood that this disclosure is not limited to the specific devices, methods, conditions or parameters described and/or shown herein, and that the terminology used herein is for the purpose of describing particular embodiments by way of example only and is not intended to be limiting of the claimed disclosure. Also, as used in the specification and including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment. It is also understood that all spatial references, such as, for example, horizontal, vertical, top, upper, lower, bottom, outer, inner, terminal (denoting position or location), left and right, posterior, anterior, and the like, are for illustrative purposes only and can be varied within the scope of the disclosure. For example, the references “superior” and “inferior” are relative and used only in the context to the other, and are not necessarily “upper” and “lower”.

Further, as used in the specification and including the appended claims, “treating” or “treatment” of a disease or condition refers to performing a procedure that may include administering one or more drugs to a patient in an effort to alleviate signs or symptoms of the disease or condition. Alleviation can occur prior to signs or symptoms of the disease or condition appearing, as well as after their appearance. Thus, treating or treatment includes preventing or prevention of disease or undesirable condition (for example, preventing the disease from occurring in a patient, who may be predisposed to the disease but has not yet been diagnosed as having it). In addition, treating or treatment does not require complete alleviation of signs or symptoms, does not require a cure, and specifically includes procedures that have only a marginal effect on the patient. Treatment can include inhibiting the disease, for example, arresting its development, or relieving the disease, for example, causing regression of the disease. For example, treatment can include reducing acute or chronic inflammation; alleviating pain and mitigating and inducing re-growth of new ligament, bone and other tissues; as an adjunct in surgery; and/or any repair procedure. Also, as used in the specification and including the appended claims, the term “tissue” includes soft tissue, ligaments, tendons, cartilage and/or bone unless specifically referred to otherwise.

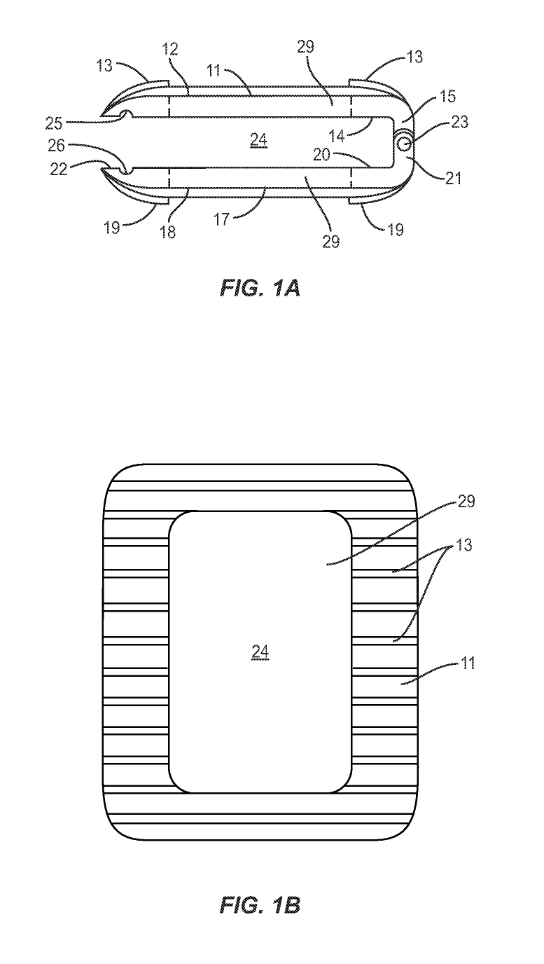

The following discussion includes a description of an expandable spinal implant and related methods of employing the expandable spinal implant in accordance with the principles of the present disclosure. Alternate embodiments are also disclosed. Reference will now be made in detail to the exemplary embodiments of the present disclosure, which are illustrated in the accompanying figures. Turning now to FIGS. 1-9, there is illustrated components of an expandable spinal implant and an implantation, or insertion, method in accordance with the principles of the present disclosure.

The components of the expandable spinal implant can be fabricated from biologically acceptable materials suitable for medical applications, including metals, synthetic polymers, ceramics and bone material and/or their composites, depending on the particular application and/or preference of a medical practitioner. For example, the components of the expandable spinal implant, individually or collectively, can be fabricated from materials such as stainless steel alloys, commercially pure titanium, titanium alloys, Grade 5 titanium (Ti-6Al-4V), super-elastic titanium alloys, cobalt-chrome alloys, stainless steel alloys, superelastic metallic alloys (for example, Nitinol, super elasto-plastic metals, such as GUM METAL® manufactured by Toyota Material Incorporated of Japan), ceramics and composites thereof such as calcium phosphate (for example, SKELITE™ manufactured by Biologix Inc.), thermoplastics such as polyaryl ether ketone including polyether ether ketone (PEEK), polyether ketone ketone and polyetherketone, carbon-PEEK composites, PEEK-BaSO₄ polymeric rubbers, polyethylene terephthalate, fabric, silicone, polyurethane, silicone-polyurethane copolymers, polymeric rubbers, polyolefin rubbers, hydrogels, semi-rigid and rigid materials, elastomers, rubbers, thermoplastic elastomers, thermoset elastomers, elastomeric composites, rigid polymers including polyphenylene, polyamide, polyimide, polyetherimide, polyethylene, epoxy, bone material including autograft, allograft, xenograft or transgenic cortical and/or corticocancellous bone, and tissue growth or differentiation factors, partially resorbable materials, such as, for example, composites of metals and calcium-based ceramics, composites of PEEK and calcium based ceramics, composites of PEEK with resorbable polymers, totally resorbable materials, such as, for example, calcium based ceramics such as calcium phosphate, tri-calcium phosphate (TCP), hydroxyapatite (HA)-TCP, calcium sulfate, or other resorbable polymers such as polylactide, polyglycolide, polytyrosine carbonate, polycaprolactone and their combinations. Various components of the expandable spinal implant may be fabricated from have material composites, including the above materials, to achieve various desired characteristics such as strength, rigidity, elasticity, flexibility, compliance, biomechanical performance, durability and radiolucency or imaging preference. The components of the expandable spinal implant, individually or collectively, may also be fabricated from a heterogeneous material such as a combination of two or more of the above-described materials. The components of the expandable spinal implant may be monolithically formed, integrally connected or include fastening elements and/or instruments, as described herein.

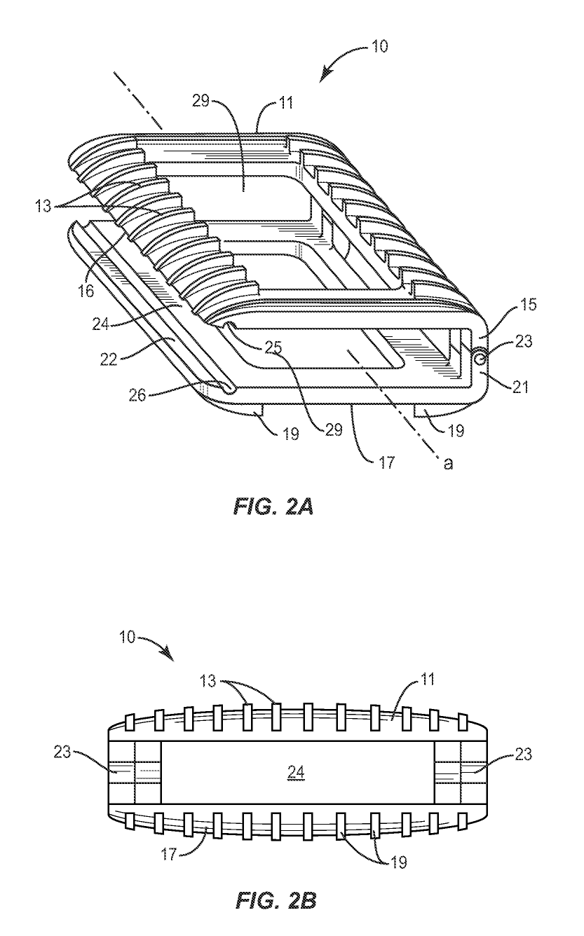

Implant portion 10 shown in FIGS. 1A, 1B, 2A and 2B in the collapsed configuration includes a first, or upper, component 11 comprising outer surface 12 possessing grooves, or teeth, 13 to enhance engagement of surface 12 with an adjacent vertebral endplate. Upper component 11 comprises an inner surface 14 and a first, or posterior terminal section 15, at one end and a second, or anterior terminal section 16 at its opposite end. Implant portion 10 further includes a second, or lower component 17 comprising outer surface 18 which, like outer surface 12 of first component 11, possesses grooves, or teeth, 19 to achieve engagement with an adjacent vertebral endplate. Lower component 17 comprises an inner surface 20 and a first, or posterior terminal section 21 at one end and a second, or anterior terminal section 22 at its opposite end.

Posterior sections 15, 21 define a posterior face of implant portion 10. Anterior sections 16, 22 define an anterior face of implant portion 10. Implant portion 10 defines a longitudinal axis a. Upper component 11 and/or lower component 17 are disposed along longitudinal axis a. It is contemplated that the axes of elongated body portions of upper component 11 and/or lower component 17 may be disposed at alternate orientations relative to longitudinal axis a, for example, perpendicular and/or angular orientations such as acute or obtuse, co-axial, parallel and/or may be offset or staggered. It is envisioned that elongated body portions of upper component 11 and/or lower component 17 may extend in alternate configurations such as, for example, arcuate, linear, offset and/or staggered.

In one embodiment, all or only a portion of upper component 11 and/or lower component 17 has a convex configuration for mating engagement with a surface of an adjacent vertebral endplate. All or only a portion of such endplate can include a concave surface. In one embodiment, all or only a portion of upper component 11 and/or lower component 17 are fabricated from an osteoconductive material. It is contemplated that the osteoconductive material may include commercially pure titanium and/or Ti-6.Al-4V. It is envisioned that all or only a portion of upper component 11 and/or lower component 17 may include, or include alternative to grooves/teeth 13, surface configurations, such as, for example, patterned, serrated, spiked, rough, threaded for connection with other instruments, arcuate, undulating, porous, semi-porous, dimpled, polished and/or textured according to the requirements of a particular application. It is contemplated that all or only a portion of upper component 11 and/or lower component 17 may have alternate cross section configurations, such as, for example, oval, oblong, triangular, rectangular, square, polygonal, irregular, uniform, non-uniform, variable and/or tapered. It is further contemplated that all or only a portion of upper component 11 and/or lower component 17 may include fastening elements such as anchors, detents and/or openings for connection to surgical instruments. It is envisioned that all or only a portion of upper component 11 and/or lower component 17 may be coated with an osteoconductive material, such as, for example. HA and/or an osteoinductive material, such as, for example, bone morphogenetic protein (BMP).

In one embodiment, all or only a portion of upper component 11 and/or lower component 17 may be fabricated from a non-metallic material, such as, for example, PEEK and are coated with a titanium alloy and/or HA.

Upper component 11 is connected in articulated engagement at its posterior section 15 with lower component 17 at its posterior section 21 for substantially vertical arcuate movement of the first component relative to the second component, the range of such movement providing an expandable distance between the first and second components.

The articulated engagement of the posterior sections 15 and 21 of upper and lower components 11 and 17 can be achieved by, for example, hinge mechanism 23, upright bendable pins or other mechanical coupling element (not shown), or a flexible connector 41, for example, one fabricated from a biocompatible flexible or elastomeric polymer as shown in FIG. 4. The pin component of hinge mechanism 23 can be fixed or slidable within a slot or groove (not shown), for example, a vertical slot, to allow some further degree of articulated engagement of the first and second components with each other. In one embodiment, upper and lower components 11 and 17 are monolithically formed such that the hinge is a living hinge connecting upper and lower components 11 and 17.

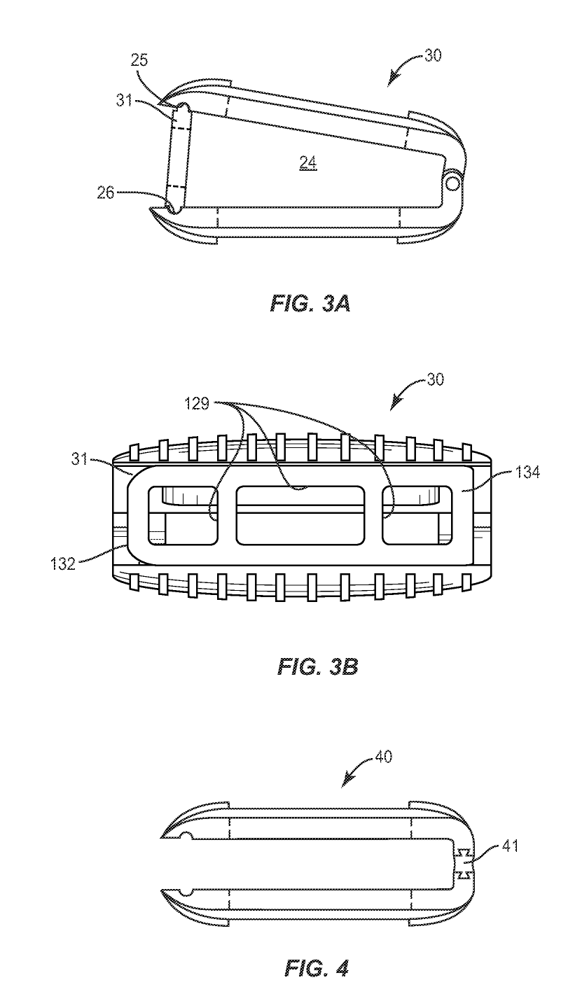

Inner surfaces 14 and 20 of upper and lower components 11 and 17 include lateral cavities, such as, for example, retaining slots 25 and 26, respectively, to accommodate the insertion of expander component 31 along a lateral pathway and/or a direct lateral approach of a body of a patient, as hereinafter described and as shown in FIGS. 3A and 3B. Retaining slots 25 and 26 are disposed adjacent the anterior face of implant portion 10. It is envisioned that retaining slot 25 and/or retaining slot 26 may be variously disposed between the anterior face and the posterior face of implant portion 10.

Retaining slots 25 and 26 have a channel configuration and are formed within surfaces 14 and 20, respectively, to a depth for receiving and retaining longitudinal end surfaces of expander component 31. Retaining slots 25 and 26 are elongated and disposed along longitudinal axis a for corresponding alignment with the orientation of the lateral pathway and/or the direct lateral approach of a body of a patient. It is contemplated that slots 25 and 26 may be disposed at alternate orientations relative to longitudinal axis a, for example, perpendicular and/or angular orientations such as acute or obtuse, co-axial, parallel and/or may be offset or staggered. It is envisioned that slots 25 and 26 may extend in alternate configurations such as, for example, arcuate, linear, offset and/or staggered.

Inner surface 14 of upper component 11 and inner surface 20 of lower component 17 are opposed to each other and define cavity 24 therebetween which can be filled, or packed, with one or more agents as more fully described, infra. When such agent(s) are those that initiate, promote and/or sustain new bone growth, one or more sides of cavity 24 will be open to vertebral surface(s) so as to achieve bony fixation. Thus, for example, one or more apertures 29 will be provided in upper and lower components 11 and 17, respectively, and, if desired, in other elements of the implant, to provide for such bony fixation occurring over time and eventually resulting in fixation of adjacent vertebrae whose endplates are in contact with outer surfaces 12 and 18 of the upper and lower components of the implant. Expander component 31 includes apertures 129, which can be filled, or packed, with one or more agents, as described. In one embodiment, the posterior face of implant portion 10 defines an opening, such as, for example, a window to facilitate medical imaging of implant 30.

Expanded implant 30 shown in FIGS. 3A and 3B possesses an expanded configuration the particular extent of which is determined by the requirements of the treatment being implemented. So, for example, in one embodiment, in a lordosing procedure carried out upon adjacent vertebrae, the lordosing angle can be set, for example, within the range of from 5 to 30 degrees. It is contemplated that the lordosing angle can be set within a range of approximately 1-45 degrees. Once the desired expanded configuration is obtained, optionally apertured expander component 31 is disposed between second terminal portions 16 and 22 of upper and lower components 11 and 17, and held in place therein by any suitable means, for example, retaining slots 25 and 26 on inner surfaces 14 and 20 of the first and second components. Expander component 31 extends between a first end 132 and a second end 134. First end 132 has a bullet nose configuration, which is tapered, to facilitate sliding engagement with slots 25, 26, for example, as expander component 31 is delivered from a direct lateral approach, for disposal between and expansion of upper and lower components 11 and 17. It is envisioned that first end 132 may include varying amounts of taper, be uniformly tapered, staggered and/or offset. In one embodiment, expander component 31 is tapered such that component 31 has a wedge configuration.

In one embodiment, expander component 31 is disposed in a locked fixation with the surface(s) of upper and lower components 11 and 17, for example, slots 25, 26. The end surfaces of expander component 31 and/or the surfaces of slots 25, 26 may include mating elements to facilitate fixation of expander component 31 with implant portion 10. It is contemplated that the mating elements can include arcuate portions, undulations, serrations and/or friction or pressure fit surfaces.

In one embodiment, spinal implant 30 is disposable in various alternate configurations between the collapsed and expanded configurations. In one embodiment, expandable spinal implant 30 can be expanded from the collapsed configuration within a range of expanded configurations with the choice of a particular expanded configuration being made to depend on the specific requirements and objectives of the treatment procedure being implemented and/or the geometry and size of expander component 31.

In one embodiment, an expandable spinal implant system is provided which includes a plurality of expandable spinal implants 30. It is contemplated that each of the plurality of expandable spinal implants 30 may have various cross section geometries and material configurations relative to other expandable spinal implants 30, and the plurality of expandable spinal implants 30 may have various orientation configurations relative to other expandable spinal implants 30. It is further contemplated that the plurality of expandable spinal implants 30 can be oriented in a side-by-side arrangement, spaced apart and/or staggered.

In assembly, operation and use, the expandable spinal implant is employed with a surgical procedure such as a direct lateral interbody fusion treatment (DLIF) of a spine of a patient including vertebrae V, intervertebral disc space I and body areas adjacent thereto, as discussed herein. The expandable spinal implant may also be employed with other surgical procedures, such as, for example, discectomy, laminotomy, laminectomy, nerve root retraction, foramenotomy, facetectomy, decompression, and spinal, nucleus or disc replacement.

For example, the expandable spinal implant can be employed with a DLIF procedure to provide height restoration between vertebral bodies for treatment of an applicable condition or injury of an affected section of a spinal column and adjacent areas within a body, such as, for example, intervertebral disc space I between the endplate of vertebrae V1 and the endplate of vertebrae V2 of vertebrae V. It is contemplated that expandable spinal implant portion 10 can be inserted within intervertebral disc space I to space apart articular joint surfaces, provide support and maximize stabilization of vertebrae V.

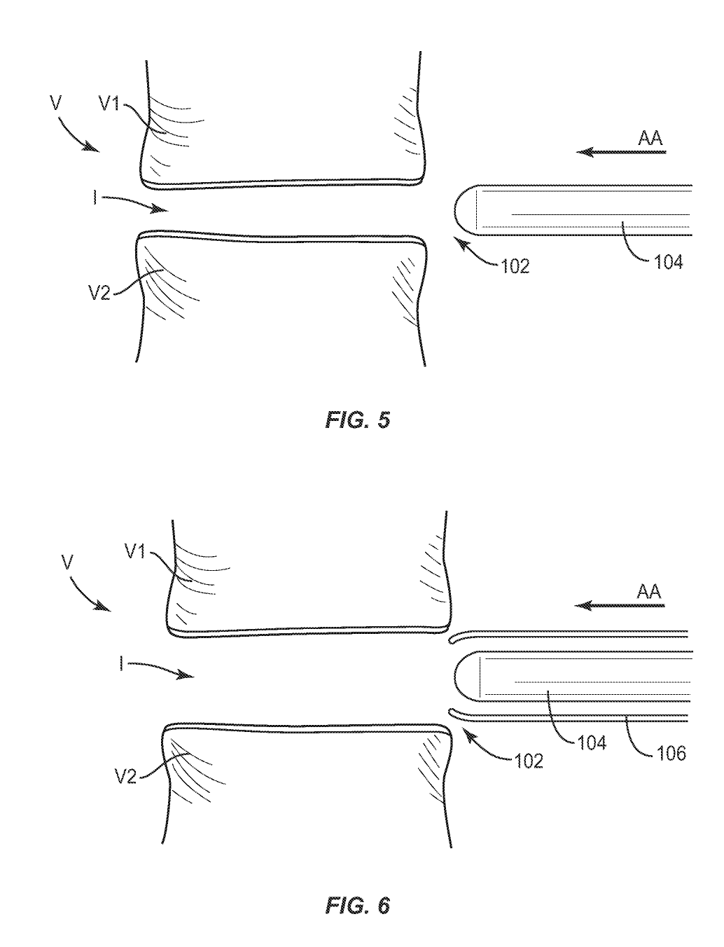

In use, as shown in FIGS. 5-9, to treat the affected section of vertebrae V, a medical practitioner obtains direct lateral access to a surgical site including vertebrae V in any appropriate manner, such as through incision and retraction of tissues. It is envisioned that expandable spinal implant 30 can be used in any existing surgical method or technique including open surgery, mini-open surgery, minimally invasive surgery and percutaneous surgical implantation, whereby vertebrae V is accessed through a mini-incision, or sleeve that provides a protected passageway to the area. Once access to the surgical site is obtained, the particular surgical procedure is performed for treating the spine disorder. Expandable spinal implant 30 is then employed to augment the surgical treatment. It is contemplated that one or all of the components of the expandable spinal implant can be delivered to the surgical site via manual manipulation and/or a free hand technique. It is further contemplated that the components of the expandable spinal implant may be inserted posteriorly, and then manipulated anteriorly and/or laterally and/or medially.

An incision is made in the body of a patient and a cutting instrument (not shown) creates a surgical pathway 102 for implantation of expandable spinal implant portion 10 within the patient body, as shown in FIG. 5. A guide instrument 104 is employed to initially distract vertebrae V1 from vertebrae V2, as manipulated in the direction of arrow AA along a direct lateral approach. A sleeve or cannula 106 is used to access intervertebral disc space I, as manipulated in the direction of arrow AA, as shown in FIGS. 5-7, and facilitate delivery and access for components of the expandable spinal implant portion 10. One or more preparation instruments (not shown) can be simultaneously or sequentially inserted within cannula 106 and disposed within intervertebral disc space I. The preparation instrument(s) can be employed to remove some or all of the disc tissue including the disc nucleus and fluids, adjacent tissues and/or bone, corticate, scrape and/or remove tissue from the surfaces of endplates of opposing vertebrae V1, V2, as well as for aspiration and irrigation of the region according to the requirements of a particular surgical application. It is envisioned that the components of the expandable spinal implant, which may include one or a plurality of expandable spinal implants 30, can be delivered to the surgical site via alternate approaches, such as, for example, delivery through the surgical pathway along a transforaminal lumbar interbody fusion approach and a posterior lumbar interbody fusion.

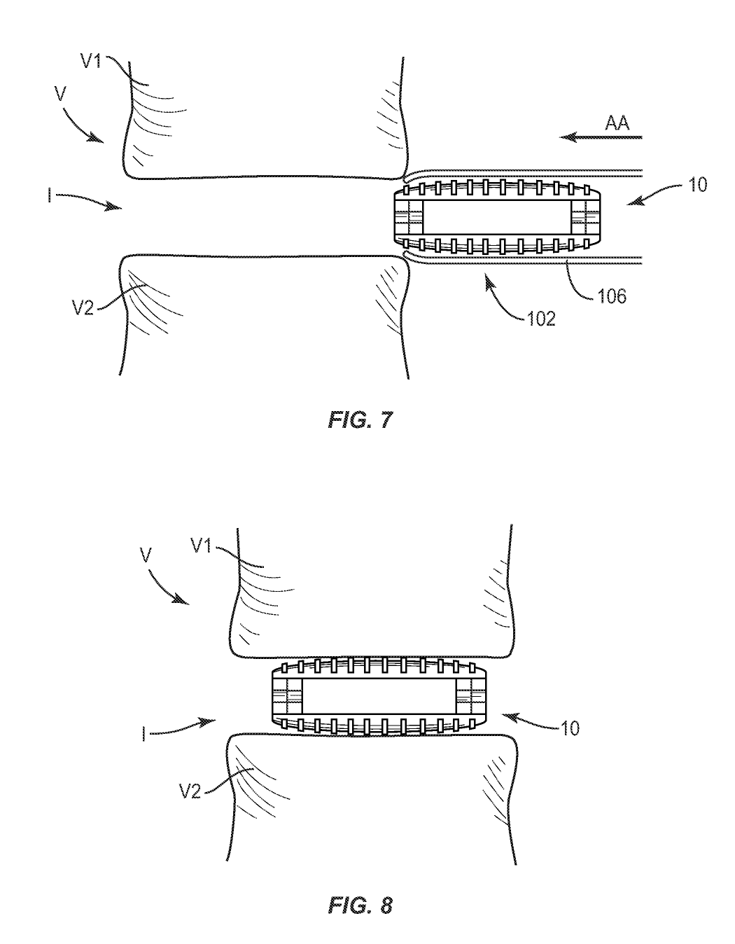

Expandable spinal implant portion 10, in the collapsed configuration, is delivered through surgical pathway 102 along the direct lateral approach, as shown in FIGS. 7 and 8, into intervertebral disc space I with a delivery instrument, or driver (not shown), via sleeve 106, as manipulated in the direction of arrow AA. The driver delivers expandable spinal implant portion 10 into the prepared intervertebral disc space I, between vertebrae V1 and vertebrae V2, according to the requirements of a particular surgical application.

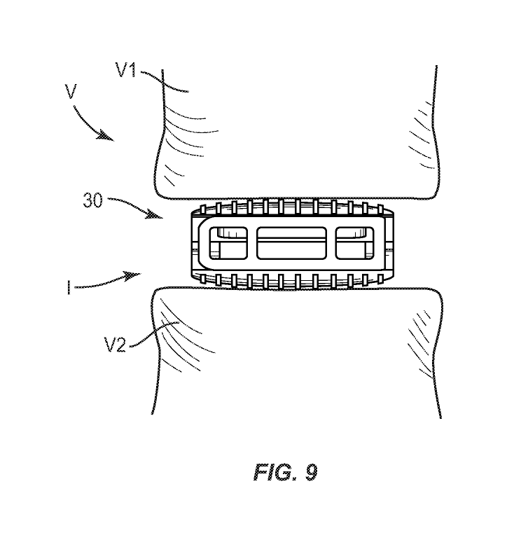

Once positioned within intervertebral space I, expandable spinal implant portion 10 is ready to be expanded to its desired expanded configuration, for example, expandable spinal implant 30, employing a suitable tool or expander instrument (not shown). For example, with implant portion 10 disposed within intervertebral space I, the implant driver maintains attachment with implant portion 10 to fix implant portion 10 within intervertebral space I. A second portion of the implant driver or a second, separate implant driver delivers expander component 31 through surgical pathway 102 along the direct lateral approach such that the longitudinal end surfaces of component 31 slide along the direction of the lateral pathway into slots 25, 26, as described above, to expand implant portion 10 to its expanded configuration, expanded spinal implant 30. The implant driver maintains orientation and position of implant portion 10 while expander component 31 is being driven into slots 25, 26 in situ. As shown in FIG. 9, expandable spinal implant 30 in its expanded configuration distracts vertebrae V1 and vertebrae V2 to restore height and decompress vertebrae V, restore lordosis, and resist subsidence of surfaces 12, 18 into vertebral endplates and migration of implant within disc space I.

Expandable spinal implant 30 remains in place within disc space I and in engagement with vertebrae V1, V2 to stabilize the area of vertebrae V in accordance with the surgical procedure. The components of expandable spinal implant 30 secure and stabilize vertebrae V in connection with the surgical procedure while preventing undesired migration of the implant. Expandable spinal implant 30 or one or more elements thereof, for example, expander component 31, can be fabricated from radiolucent materials such as polymers. Radiomarkers may be included for identification under x-ray, fluoroscopy, CT or other imaging techniques.

In one embodiment, the expandable spinal implant herein includes at least one agent, which may be disposed, packed or layered within, on or about the components amid/or surfaces thereof. For example, the at least one agent is configured for inclusion within cavity 24. The at least one agent can include bone growth promoting material, such as, for example, bone graft. The bone graft can be a particulate material, which may include an osteoconductive material such as HA and/or an osteoinductive agent such as a BMP to enhance bony fixation of expandable spinal implant 30 with the adjacent vertebrae V.

It is contemplated that the bone graft may include therapeutic polynucleotides or polypeptides. It is further contemplated that the bone graft may include biocompatible materials, such as, for example, biocompatible metals and/or rigid polymers, such as, titanium elements, metal powders of titanium or titanium compositions, sterile bone materials, such as allograft or xenograft materials, synthetic bone materials such as coral and calcium compositions, such as hydroxyapatite, calcium phosphate and calcium sulfite, biologically active agents, for example, gradual release compositions such as by blending in a bioresorbable polymer that releases the biologically active agent or agents in an appropriate time dependent fashion as the polymer degrades within the patient. Suitable biologically active agents include, for example, BMP, Growth and Differentiation Factors proteins (GDF) and cytokines.

It is envisioned that the agent may include one or a plurality of therapeutic agents and/or pharmacological agents for release, including sustained release, to treat, for example, pain, inflammation and degeneration. The agents may include pharmacological agents, such as, for example, antibiotics, anti-inflammatory drugs including but not limited to steroids, anti-viral and antiretroviral compounds, therapeutic proteins or peptides, therapeutic nucleic acids (as naked plasmid or a component of an integrating or non-integrating gene therapy vector system), and combinations thereof.

The agent may also include analgesics or anesthetics such as acetic acid derivatives, COX-2 selective inhibitors, COX-2 inhibitors, enolic acid derivatives, propionic acid derivatives, salicylic acid derivatives, opioids, opioid/nonopioid combination products, adjuvant analgesics, and general and regional/local anesthetics.

The agent may also include antibiotics such as, for example, amoxicillin, beta-lactamases, aminoglycosides, beta-lactam (glycopeptide), clindamycin, chloramphenicol, cephalosporins, ciprofloxacin, erythromycin, fluoroquinolones, macrolides, metronidazole, penicillins, quinolones, rapamycin, rifampin, streptomycin, sulfonamide, tetracyclines, trimethoprim, trimethoprim-sulfamthoxazole, and vancomycin.

The agent may also include immunosuppressives agents, such as, for example, steroids, cyclosporine, cyclosporine analogs, cyclophosphamide, methylprednisone, prednisone, azathioprine, FK-506, 15-deoxyspergualin, prednisolone, methotrexate, thalidomide, methoxsalen, rapamycin, leflunomide, mizoribine (Bredinin™), brequinar, deoxyspergualin, and azaspirane (SKF 105685), Orthoclone OKT™ 3 (muromonab-CD3). Sandimmune™, Neoral™, Sangdya™ (cyclosporine), Prograf™ (FK506, tacrolimus), Cellcept™ (mycophenolate motefil, of which the active metabolite is mycophenolic acid), Imuran™ (azathioprine), glucocorticosteroids, adrenocortical steroids such as Deltasone™ (prednisone) and Hydeltrasol™ (prednisolone), Folex™ and Mexate™ (methotrxate), Oxsoralen-Ultra™ (methoxsalen) and Rapamuen™ (sirolimus).

It will be understood that various modifications may be made to the embodiments disclosed herein. Therefore, the above description should not be construed as limiting, but merely as exemplification of the various embodiments. Those skilled in the art will envision other modifications within the scope and spirit of the claims appended hereto.

What is claimed is:

1. An expandable spinal implant comprising:
a first component comprising outer and inner surfaces, a first terminal section at one end of the first component and a second terminal section at its other end, the first component defining a lateral slot within the inner surface;
a second component comprising outer and inner surfaces, a first terminal section at one end of the second component and a second terminal section at its other end, the inner surface of the second component defining a lateral slot, the first terminal section of the first component being connected with the first terminal section of the second component for substantially vertical arcuate movement of the first component relative to the second component, the range of such movement providing an expandable distance between the second terminal sections of the first and second components,
wherein the first terminal sections of the first and second components define a posterior face of the implant and the second terminal sections of the first and second components define a anterior face of the implant; and
an expander component disposable within the lateral slots of the first and second components in a configuration for maintaining a predetermined distance between the second terminal sections of the first and second components.

2. The expandable spinal implant of claim 1 wherein the outer surfaces of the first and second components are textured for enhanced engagement of such surfaces with the endplates of adjacent vertebrae.

3. The expandable spinal implant of claim 1 wherein the outer surfaces of the first and second components include grooves or teeth for enhanced engagement of such surfaces with the endplates of adjacent vertebrae.

4. The expandable spinal implant of claim 1 wherein the opposed inner surfaces of the first and second components define a cavity and the first and second components include one or more apertures.

5. The expandable spinal implant of claim 1 wherein the opposed inner surfaces of the first and second components define a cavity containing at least one agent and the first and second components possess one or more apertures.

6. The expandable spinal implant of claim 1 wherein the articulated engagement of the first and second components is provided by a hinge.

7. The expandable spinal implant of claim 1 wherein articulated engagement of the first and second components is provided by a flexible connector.

8. The expandable spinal implant of claim 1 wherein articulated engagement of the first and second components is provided by a flexible connector fabricated from a biocompatible flexible or elastomeric polymer.

9. The expandable spinal implant of claim 1 wherein one or more components are fabricated from biocompatible metal and/or one or more components are fabricated from biocompatible ceramic, polymer or bone.

10. The expandable spinal implant of claim 1 wherein one or more components are fabricated from biocompatible radiolucent material.

11. The expandable spinal implant of claim 1 wherein the first and second components are fabricated from biocompatible metal and the expander component is fabricated from a biocompatible radiolucent polymer.

12. The expandable spinal implant of claim 1 wherein the first and second components are fabricated from titanium metal or titanium metal alloy and the expander component is fabricated from polyether ether ketone.

13. The expandable spinal implant of claim 1 wherein the lateral slots are disposed adjacent the anterior face and the expander component comprises longitudinal end surfaces configured for slidable engagement with the lateral slots.

14. A lordosing implant comprising:
an upper component comprising an outer, vertebral end plate-engaging surface, an inner surface defining a lateral cavity, a posterior section at one end of the component and an anterior section at the other end thereof;
a lower component comprising an outer, vertebral endplate-engaging surface, an inner surface facing the inner surface of the upper component, the inner surface of the lower component defining a lateral cavity, the inner surfaces further defining a cavity therebetween, posterior section at one end of the component and an anterior section at the other end thereof,
the posterior section of the upper component being connected by a flexible member to the posterior section of the lower component for substantially vertical arcuate movement of the upper component relative to the lower component, the range of such movement extending from a first, collapsed configuration of the upper and lower components to a second, lordosing configuration of the upper and lower components; and
an expander component disposable within the lateral cavities,
wherein the implant is disposable within an intervertebral space such that the lateral cavities are aligned along a lateral pathway of a body such that the expander component maintains a predetermined distance between the anterior sections corresponding to a desired lordosing configuration of the upper and lower components.

15. The lordosing implant of claim 14 wherein the outer, vertebral endplate-engaging surfaces of the upper and lower components possess grooves or teeth for enhanced engagement of the surfaces with the vertebral endplates.

16. The lordosing implant of claim 15 wherein the upper and lower components include one or more apertures and the cavity contains at least one agent.

17. The lordosing implant of claim 16 wherein at least one component is fabricated from biocompatible metal and/or at least one member is fabricated from biocompatible ceramic, polymer or bone.

18. The lordosing implant of claim 14 wherein the lateral cavities are disposed within the anterior sections.

19. A method for treating a spine, the method comprising the steps of:
a) providing an expandable spinal implant portion comprising:
a first component comprising outer and inner surfaces, a first terminal section at one end of the first component and a second terminal section at its other end; and
a second component comprising outer and inner surfaces, a first terminal section at one end of the second component and a second terminal section at its other end, the first terminal section of the upper component being connected in articulated engagement with the first terminal section of the lower component for substantially vertical arcuate movement of the first component relative to the second component, the range of such movement providing an expandable distance between the second terminal sections of the first and second components;
b) introducing the expandable spinal implant portion along a direct lateral pathway of a body within an intervertebral space; and
c) introducing an expander component along the direct lateral pathway between the second terminal sections of the first and second components to expand the distance between the second terminal sections of the first and second components of the expandable spinal implant portion by a predetermined distance, the expander component being sized for maintaining the predetermined distance between the second terminal portions of the first and second components.

20. A method of claim 19 wherein in step (a), the expandable spinal implant portion comprises:
an upper component comprising an outer, vertebral endplate-engaging surface, an inner surface, a posterior section at one end of the component and an anterior section at the other end thereof; and
a lower component comprising an outer, vertebral endplate-engaging surface, an inner surface facing the inner surface of the upper component and defining a cavity therebetween, a posterior section at one end of the component and an anterior section at the other end thereof, the posterior section of the upper component being connected by a hinge or flexible member to the posterior section of the lower component for substantially vertical arcuate movement of the upper component relative to the lower component, the range of such movement extending from a first, collapsed configuration of the upper and lower components to a second, lordosing configuration of the upper and lower components; and
in step (c), the anterior sections of the upper and lower components of the expandable spinal implant are expanded by a predetermined distance corresponding to a desired lordosing configuration.
